(19) United States
(12) Patent Application Publication
(10) Pub. No.: US 2012/0316144 A1
(43) Pub. Date: Dec. 13, 2012

(54) ANTIFUNGAL AGENT

(76) Inventor: Hideo Minobe, Munakata-shi (JP)

(21) Appl. No.: 13/576,061

(22) PCT Filed: Apr. 30, 2010
(86) PCT No.: PCT/JP2010/057658

§ 371 Date: Aug. 22, 2012

(30) Foreign Application Priority Data
Feb. 25, 2010 (JP) 2010-039855

(51) Int. Cl. A61K 31/122; A61P 31/10; A61K 31/575

(52) U.S. Cl. 514/171; 514/691

(57) ABSTRACT

An antifungal agent including a mastic normal-hexane extract containing at least one of triterpenes expressed by the following chemical formulas 1 to 6;

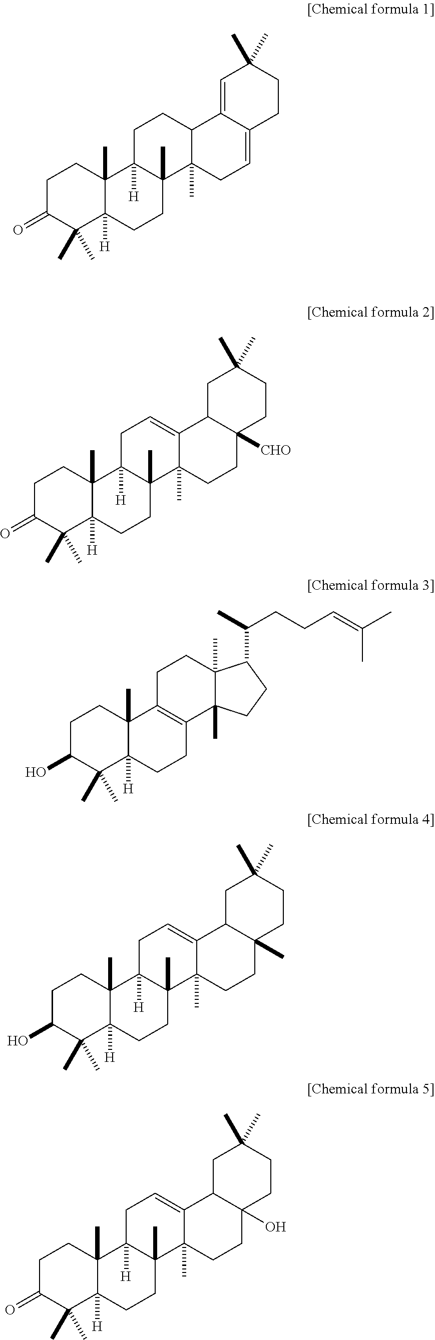

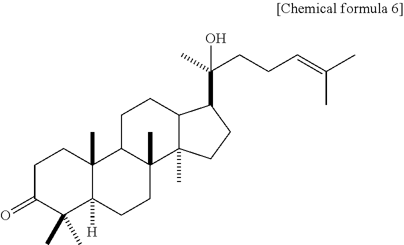

and squalene.

### BACKGROUND OF THE INVENTION

The present invention relates to an antifungal agent. Specifically, the present invention relates to an antifungal agent for treatment of fungal infections and for prevention of fungal infections.

Funguses cause infections in humans, animals, and the like, to cause diseases.

For example, a fungus causes a superficial mycosis in human skin or the like. Cleaning of hands and fingers, and affected areas are carried out with ethanol for disinfection, disinfectants, and fungicides on the nursing-care and medical fronts. However, a high frequency of cleaning is very likely to damage skin such as causing skin to become rough, stinging wounds, or worsening of dermatitis.

Further, on the veterinary care front, Malassezia infections in dogs are observed in many cases.

Further, in recent years, opportunistic infections of which patients having cancerous diseases such as leukemia or malignant lymphoma, or underlying diseases such as metabolic disturbances, users of steroids or immunosuppressants, or compromised hosts whose resistance to infection due to radiation are infected with attenuated pathogens which normally do not show pathogenicity, have become problematic, and have caused concerns on the medical front.

As antifungal agents generally used on the market, there are azole antifungal agents, polyene antifungal agents, and candin antifungal agents.

For example, in Japanese Published Unexamined Patent Application No. 2000-186037, there is disclosed an antifungal agent containing pyrrolnitrin and clotrimazole as a medical agent that contains two azole antifungal agents.

However, because funguses and cells of mammals such as humans are similar in cellular structure, antifungal agents are low in selective toxicity acting only on funguses. As a result, because antifungal agents are also damaging to cells of mammals such as humans, and further their effective doses and toxic doses are approximate to one another, in particular, when an artificially synthetic antifungal agent is used, it is easy to cause side effects such as nausea, emesis, fever, renal damage, and the like.

In addition, in recent years, many people would like to ingest and use, not artificially synthesized compounds, but naturally-derived substances as drugs dosed to patients, and soaps and shampoos used in daily life.

The present invention has been made in consideration of the above points, and an object of the present invention is to provide an antifungal agent which has a high antifungal activity, and is safe to use even for the human body.

### SUMMARY OF THE INVENTION

In order to achieve the above-described object, the antifungal agent of the present invention contains a mastic extract and squalene.

Further, in the antifungal agent of the present invention, the mastic extract is preferably a mastic normal-hexane extract.

Further, in the antifungal agent of the present invention, the mastic normal-hexane extract preferably contains at lease one of triterpenes expressed by the following chemical formulas 1 to 6.

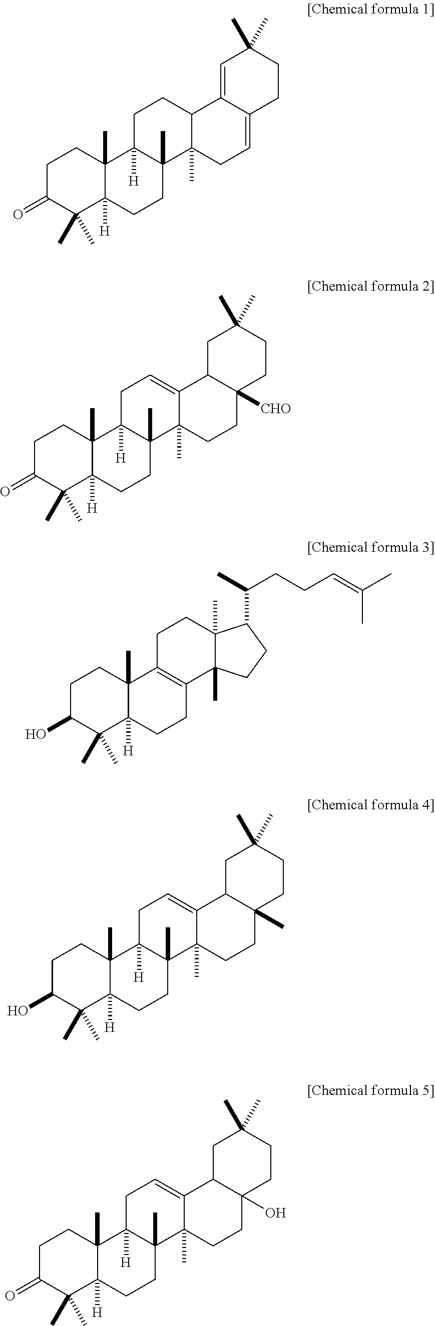

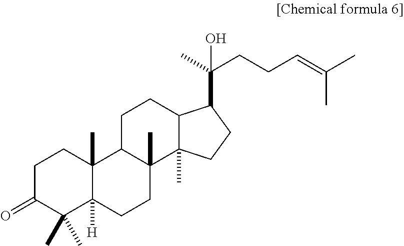

The antifungal agent according to the present invention has a high antifungal activity and is safe to use even for the human body.

### DETAILED DESCRIPTION OF THE PREFERRED EMBODIMENTS

Squalene (molecular formula C₃₀H₅₀) used for the antifungal agent of the present invention is an unsaturated hydrocarbon (unsaturated isoprenoid) at a degree of purity of 99% or more, and a simple lipid having six double bonds. Squalene is contained in deep-sea shark liver oil at approximately 70 to 90%, and commercially available squalene is extracted from shark liver oil.

Further, squalene is a substance which induces a derivative of the basic molecular formula (C₅H₈)ₙ whose building block is isoprene (molecular formula C₅H₈), i.e., a triterpene of terpene group, to serve an important role in life such as having antibacterial and bactericidal activities that are characteristic to terpene.

Further, the behaviors of activated squalene are as follows.

(1) Boiling point: 252 to 254° C. (5 mmHg), 262 to 264° C. (10 mmHg), 330° C. (normal pressures).

(2) Freezing point: −20° C., transparency maintained even after keeping it for one hour. Frozen into a white wax clump at −75° C.

(3) Iodine number: approximately 385 to 420. Normally 371.

(4) The fact that an iodine number is high means that it has many double bonds, which can be said to be biochemically active.

On the other hand, mastic used for the antifungal agent of the present invention is whitish yellow transparent and granular resin which is taken and extracted from Pistacia lentiscus LINNE, that is an anacardiaceous shrub, and contains mastica dienoic acid as a major ingredient.

Further, a mastic tree secretes a highly thick fluid as self-protection against a foreign invader or an epidemic. It has been found from studies in recent years that its tree sap has a biological protection function such as immunoproliferative potential, an antibacterial action, vital function adjusting power, and skin protection power.

Further, in Europe and the United States, in the healthcare field and the medical field, it has been reported that mastic has various types of plague preventive and therapeutic effects, and palliation actions of symptoms as follows.

(1) Internal medicine: Treatment and prevention of gastric ulcers and duodenal ulcers, a blood pressure reduction in hypertension, an antihyperglycemic action in diabetes, a hypocholesterolemic action, enhancement of immune system, preventive effects on various types of cancers, being cholagogue, a gallstone dissolving action, prevention of tongue coating, alleviation of pains of gout and rheumatism, and the like.

(2) Dentistry: Prevention and treatment of alveolar pyorrhea, prevention and treatment of periodontal diseases, inhibition of bacterial plaque, prevention of gum bleeding, and muscular strengthening.

(3) Surgical therapy and prophylaxis, acceleration of wound healing, healing of cutaneous ulceration such as a pressure ulcer.

Mastic itself is of a hard resinous form, which has a poor biological absorption, and for that reason, in Europe and the United States, mastic has not been clinically used for treatment and prevention of fungal infections.

Therefore, with respect to improvements in symptoms for which improvement effects cannot be obtained by a single application of mastic, a comprehensively newer and more pronounced antifungal activity could be obtained by use of a combination of squalene and extracted ingredients of mastic. However, combinatorial compounding of product ingredients for medicinal and health prevention of mastic has not been studied at all thus far.

The compounding amounts of mastic, or mastic extracts, and squalene which are contained in the antifungal agent of the present invention are not particularly limited. However, for example, squalene of 1 mg to 1000 mg and mastic of 1 mg to 1000 mg may be the compounding amounts.

Further, it is possible to manufacture an antifungal agent having higher antimycotic action such that one ingredient or several ingredients are selected from among Hinokitiol, herbs, Phellodendron amurense (Kihada), Coptis japonica (Ouren), dried roots of Scutellaria baicalensis(Wogon), Glycyrrhiza, Pseudoginseng, Panax ginseng, crude drug extract, Thujopsis dolabrata oil, jojoba oil, plant essential oil, horse oil, lard, eicosapentaenoic acid, docosahexaenoic acid, perilla oil, garlic oil, synthetic vitamin E, natural vitamin E, γ-orizanol, β-carotene, vitamin B1, vitamin B2, vitamin B6, vitamin 12, vitamin C, polyphenol, lycopene, extract of cultures of mushroom mycelium, extract of cultures of mushroom fruiting body, Ganoderma lucidum, Agaricus extract, sasa veitchii extract, Grifola frondosa extract, chlorella, Spirulina, tannin, alginic acid, catechin extract, glycyrrhizin, propolis, ginkgo leaf extract, Panax ginseng extract, Panax quinquefolius extract, Eleutherococcus senticosus extract, Salvia miltiorrhiza leaf extract, aloe extract, chitosan, inositol, peptide, ceramide, niacin, total isoflavone, pantothenic acid, biotin, total carotenoid, selenium, zinc, chrome, collagen, chondroitin sulfuric acid, mucopolysaccharide-protein, calcium, royal jelly, millet, silica, saw palmetto, turmeric extract, Silybum marianum extract, Garcinia cambogia extract, isopropanol, sodium hyaluronate, chlorhexidine gluconate, povidone-iodine, ethanol, benzalkonium cetyl phosphate, triclosan, chloroxylenol, isopropyl methylphenol, benzalkonium chloride, and the like, to be compounded into the antifungal agent of the present invention.

Further, the antifungal agent of the present invention may further contain one ingredient or several ingredients selected from among coenzyme Q10, α-lipoic acid, vitamin P, vitamin E, γ-orizanol, β-carotene, tannin, vitamin C, and heme iron as an antioxidant degradation control agent.

Further, as a drug formulation of the antifungal agent of the present invention, an encapsulated formulation, a tablet, or the like can be cited.

Further, the antifungal agent of the present invention may contain an emulsifier or a plasticizer, to be a solid form, a gel form, a cream form, an ointment form, or a liquid form.

Here, an example of an emulsifier is a palm oil, and an example of a plasticizer is beeswax.

Further, an amount of beeswax added to the antifungal agent of the present invention may be 50 mg to 600 mg, and an amount of refined palm oil may be 50 mg to 600 mg.

Moreover, provided that an amount of squalene is 50 mg to 1000 mg, it is possible to, not only increase the concentration of mastic extracts dissolved into the squalene, to increase the absorption thereof, but also improve the antifungal effect and the moisturizing effect. In particular, the case where the antifungal agent of the present invention is used as a cleaning agent and a drug for external use, that point should be noted.

### First Example

### Separation of Antifungal Active Ingredients of Mastic

After mastic resin (20.5 g) was pounded in a mortar, the pounded mastic resin was divided with solutions of normal-hexane (400 mL) and methanol (400 mL).

Then, after the normal-hexane layer and the methanol layer were separated with a separating funnel, the same separation operation with respect to methanol layer was carried out three times with normal-hexane (300 mL).

Next, vacuum condensation of the normal-hexane layer (approximately 900 mL) and the methanol layer (approximately 650 mL) was carried out at 40 degrees by an evaporator, to obtain a mastic normal-hexane extract (11.5 g) and a methanol extract (8.0 g).

Moreover, column chromatography (inner diameter of 2.5 cm, and height of 30 cm) with silica gel (approximately 70 g, Silica gel 60 manufactured by Merck KGaA) was carried out with respect to the mastic normal-hexane extract (1.02 g).

That is, as a column eluting solvent, normal-hexane/ethyl acetate (loll) of 1.1 L was used, and next, normal-hexane/ethyl acetate (7/1) of 0.8 L was used.

Next, an eluate of approximately 10 mL was fractionated in the test tube, and the eluted substance was monitored by a thin-layer chromatography (TLC=Thin-Layer Chromatography).

Then, the fractionated eluate was condensed by an evaporator, to obtain nine fractions [Fr. 1 (32.3 mg), Fr. 2 (1.7 mg), Fr. 3 (38.3 mg), Fr. 4 (37.1 mg), Fr. 5 (26.4 mg), Fr. 6 (22.8 mg), Fr. 7 (19.0 mg), Fr. 8 (34.8 mg), Fr. 9 (522.7 mg)].

Moreover, Fr. 5 (26.4 mg) was separated by a reverse HPLC (=High-Performance Liquid Chromatography), to obtain Fr. 5-1 (5.3 mg) and Fr. 5-2 (2.1 mg). Here, the condition was that the used column was a COSMOSIL (registered trademark) AR-IIC₁₈ (the inner diameter of 4.6 mm, the length of 250 mm) manufactured by NACALAI TESQUE, INC., the used solvent was methanol, the current velocity was 1.0 mL/min, and the detection was at UV220 nm).

### Second Example

### Identification of Antifungal Active Ingredient of Mastic

With respect to Fr. 1, Fr. 3, Fr. 5-1, Fr. 5-2, Fr. 6, and Fr. 8 obtained by fractionation, structural analyses were carried out by use of a gas chromatograph mass spectrometer (QP5050A manufactured by SHIMADZU CORPORATION) and a nuclear magnetic resonance apparatus (NMR=Nuclear Magnetic Resonance) (Unity 600 manufactured by Varian Medical Systems, Inc.). The results are shown below.

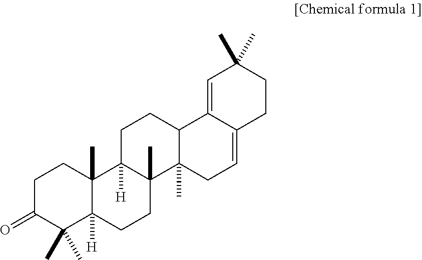

As a result of the structural analysis of Fr. 1, it was found that it is 3-oxo-28-norolean-16,18-diene, which is expressed by the above-described chemical formula 1. Further, the structural analysis data of Fr. 1 are as follows.

EIMS (relative intensity) (m/z): 408 (M⁺), 393 (M-15), 207;

¹H NMR (CDCl₃, 600 MHz): 0.77 (3H, s), 0.95 (3H, s), 0.96 (3H, s), 0.97 (6H, s), 1.01 (6H, s), 1.07 (3H, s), 5.15 (1H, brs), 5.28 (1H, m);

¹³C-NMR (CDCl₃, 150 MHz): 14.5, 16.3, 16.6, 19.5, 21.0, 21.1, 25.1, 26.9, 27.9, 28.8, 29.8, 31.0, 32.6, 32.9, 33.8, 34.1, 36.8, 37.4, 38.0, 39.7, 40.1, 41.3, 47.3, 49.8, 54.8, 120.2, 131.9, 132.5, 134.9, 218.1

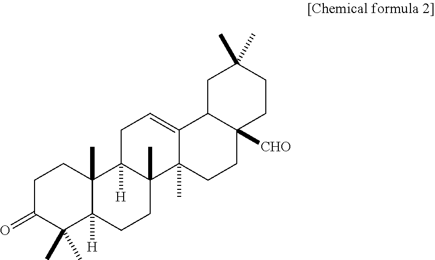

As a result of the structural analysis of Fr. 3, it was found that it is olean-12-en-3,28-dione (or oleanoic aldehyde), which is expressed by the above-described chemical formula 2. Further, the structural analysis data of Fr. 3 are as follows.

EIMS (relative intensity) (m/z): 438 (M⁺), 409 (M-29), 203 (base peak);

¹H-NMR (CDCl₃, 600 MHz): 0.77 (311, s), 0.89 (311, s), 0.90 (311, s), 1.02 (6H, s), 1.06 (3H, s), 1.13 (3H, s), 5.33 (1H, brs), 9.37 (1H, s);

¹³C-NMR (CDCl₃, 150 MHz): 15.1, 17.0, 19.6, 21.5, 22.1, 23.4, 23.5, 25.4, 26.5, 26.7, 27.7, 30.6, 32.3, 33.0, 33.2, 34.1, 36.7, 39.2, 39.6, 40.5, 41.8, 45.6, 46.8, 47.4, 49.1, 55.3, 123.0, 143.1, 207.3, 218.0

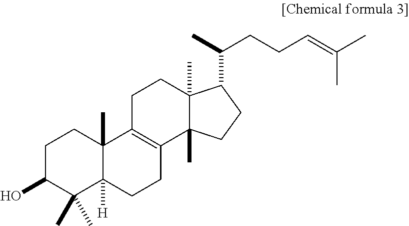

As a result of the structural analysis of Fr. 5-1, it was found that it is Tirucallol, which is expressed by the above-described chemical formula 3. Further, the structural analysis data of Fr. 5-1 are as follows.

EIMS (m/z): 426 (M⁺), 411 (M-15), 393 (M-15-18), 69 (base peak);

¹H-NMR (CDCl₃, 600 MHz): 0.74 (311, s), 0.78 (3H, s), 0.85 (311, brs), 0.89 (311, d), 0.94 (311, s), 0.95 (3H, s), 0.98 (3H, s), 1.58 (3H, s), 1.66 (3H, s), 3.21 (1H, dd), 5.08 (1H, brd);

¹³C-NMR (CDCl₃, 150 MHz): 15.4, 15.5, 17.6, 18.7, 18.9, 20.1, 21.5, 24.4, 24.9, 25.7, 27.7, 28.0, 28.1, 29.8, 30.8, 35.3, 36.3, 36.4, 37.3, 38.9, 44.1, 50.0, 50.1, 51.0, 79.0, 125.3, 130.6, 133.2, 133.8

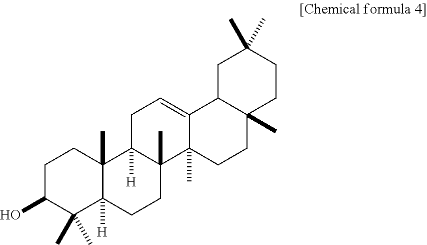

As a result of the structural analysis of Fr. 5-2, it was found that it is β-amylin, which is expressed by the above-described chemical formula 4. Further, the structural analysis data of Fr. 5-2 are as follows.

EIMS (m/z): 426 (M⁺), 411 (M-15), 393 (M-15-18), 218 (base peak), 203;

¹H-NMR (CDCl₃, 600 MHz): 0.77 (3H, s), 0.82 (3H, s), 0.85 (6H, brs), 0.92 (3H, s), 0.95 (3H, s), 0.98 (3H, s), 1.11 (3H, s), 3.20 (1H, dd, 4.6, 11.2 Hz), 5.16 (1H, brt)

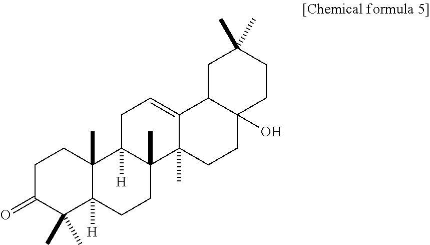

As a result of the structural analysis of Fr. 6, it was found that it is 3-oxo-17-hydroxy-norolean-12-ene, which is expressed by the above-described chemical formula 5. Further, the structural analysis data of Fr. 6 are as follows.

EIMS (m/z): 426 (M⁺), 409 (M-17), 394, 281, 253, 207, 202, 55 (base peak);

¹H-NMR (CDCl₃, 600 MHz): 0.88 (3H, s), 0.95 (3H, s), 0.97 (3H, s), 1.04 (3H, s), 1.04 (3H, s), 1.08 (3H, s), 1.12 (3H, s), 5.20 (1H, brs);

¹³C-NMR (CDCl₃, 150 MHz): 15.0, 17.0, 19.6, 21.5, 23.3, 23.6, 23.9, 25.3, 25.5, 26.5, 31.0, 31.2, 32.4, 32.5, 34.2, 36.0, 36.8, 39.1, 39.5, 41.7, 42.8, 47.0, 47.4, 48.3, 55.4, 84.2, 122.3, 143.7.217.7

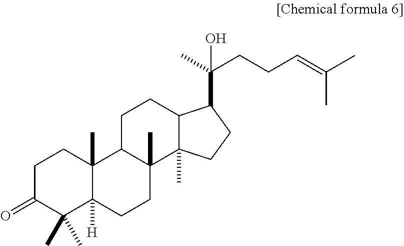

As a result of the structural analysis of Fr. 8, it was found that it is 3-oxo-20-hydroxy-dammar-24-ene, which is expressed by the above-described chemical formula 6. Further, the structural analysis data of Fr. 8 are as follows.

EIMS (m/z): 424 (M⁺-18), 409, 355, 109 (base peak); ¹H-NMR (CDCl₃, 600 MHz): 0.87 (3H, s), 0.93 (3H, s), 0.98 (3H, s), 1.02 (311, s), 1.06 (3H, s), 1.13 (3H, s), 1.61 (3H, s), 1.67 (3H, s), 5.11 (1H, m);

¹³C-NMR (CDCl₃, 150 MHz): 15.2, 16.0, 16.3, 17.7, 19.7, 21.0, 22.0, 22.6, 24.8, 25.5, 25.7, 26.7, 27.5, 31.2, 34.1, 34.6, 36.8, 39.9, 40.3, 40.5, 42.4, 47.4, 49.8, 50.0, 50.3, 55.4, 75.4, 124.7, 131.6, 218.0

### Third Example

### Antibacterial Test of Mastic Ingredient for Drug Resistance Bacterium and Fungus

First, actions on drug resistance bacterium, of mastic normal-hexane extracts (hereinafter referred to as “MH”) and methanol extracts (hereinafter referred to as “MM”) were confirmed.

Further, the used fungus bodies are the following four fungus bodies that cause concern on the medical front.

(1) Methicillin-resistant Staphylococcus aureus(MRSA)
(2) Vancomycin-resistant enterococcus (VRE)
(3) Escherichia coli bacterium (E. coli)
(4) Pseudomonas aeruginosa (P. aeruginosa)

MH and MM were respectively dissolved in acetone. Paper disks for antibiotic assay (with a thickness of 8 mm, ADVANTEC (registered trademark) were impregnated with the obtained MH solution, to prepare a disk to be tested impregnated with the MH solution of 5 mg/disk, and a disk to be tested impregnated with the MH solution of 20 mg/disk.

Further, in the same manner, paper disks for antibiotic assay (with a thickness of 8 mm, ADVANTEC (registered trademark) were impregnated with the obtained MM solution, to prepare a disk to be tested impregnated with the MM solution of 5 mg/disk, and a disk to be tested impregnated with the MM solution of 20 mg/disk.

Further, as standard controls, paper disks for antibiotic assay (with a thickness of 8 mm, ADVANTEC (registered trademark) were impregnated with acetone only, to prepare a disk to be tested impregnated with acetone of 5 mg/disk, and a disk to be tested impregnated with acetone of 20 mg/disk.

Then, the respective disks to be tested were vacuum-dried to eliminate the acetone.

Further, the respective fungus bodies of (1) to (4) prepared to be approximately 10⁹ CFU/ml were applied to ordinary agar media. Further, in the same manner, the respective fungus bodies of (1) to (4) prepared to be approximately 10⁹ CFU/ml were applied to Mueller-Hinton media.

Next, the respective disks to be tested were placed on the ordinary agar media, and cultured for two days under an aerobic condition at 37° C. Further, in the same manner, the respective disks to be tested were placed on the Mueller-Hinton media, and cultured for two days under an aerobic condition at 37° C. Then, the diameters (mm) of antiproliferative circles formed around the respective disks to be tested were measured. The results are shown in Table 1 and Table 2.

TABLE 1
<Table 1> Ordinary agar media

| | Extracts prepared to be respective dosages | | | |
|---|---|---|---|---|
| | MH (5 mg) | MH (20 mg) | MM (5 mg) | MM (20 mg) |
| MRSA | — | 13 | — | — |
| VRE | — | — | — | — |
| E. coli | — | — | — | — |
| P. aeruginosa | — | — | — | — |

(“—” denotes no antibacterial activity)

TABLE 2
<Table 2> Mueller-Hinton media

| | Extracts prepared to be respective dosages | | | |
|---|---|---|---|---|
| | MH (5 mg) | MH (20 mg) | MM (5 mg) | MM (20 mg) |
| MRSA | — | 16 | — | — |
| VRE | — | — | — | — |
| E. coli | — | — | — | — |
| P. aeruginosa | — | — | — | — |

(“—” denotes no antibacterial activity)

As is clear from Table 1 and Table 2, the disk to be tested impregnated with the mastic normal-hexane extract (MH) of 20 mg showed the anti-drug resistance bacterium action only on the MRSA. However, no antibacterial activity was confirmed in the methanol extracts.

### Fourth Example

### Synergetic Effect of Antifungal (Malassezia) Activity of Mastic Normal-Hexane Extract and Squalene

Mastic normal-hexane extract (MH) was dissolved in acetone. Then, paper disks for antibiotic assay which are the same as the paper disks for antibiotic assay used in the third example were impregnated with the obtained MH solution, to prepare a disk to be tested impregnated with the MH solution of 5 mg/disk, a disk to be tested impregnated with the MH solution of 10 mg/disk, and a disk to be tested impregnated with the MH solution of 20 mg/disk. These disks to be tested were prepared two each, respectively.

Further, the S solution, which was obtained by dissolving squalene (S) in acetone, was added to each of some disks to be tested prepared in this way by 25 mg/disk, to prepare disks of mixture of the mastic normal-hexane extract and squalene (MH+S) as well.

Further, as standard controls, paper disks for antibiotic assay which are the same as the paper disks for antibiotic assay used in the third example were impregnated with the S solution of 25 mg/disk, to prepare a disk to be tested.

Then, the respective disks to be tested were vacuum-dried to eliminate the acetone.

Further, the Malassezia prepared to be approximately 10⁶ CFU/ml was applied to the Mueller-Hinton media.

Next, the respective disks to be tested were placed on the Mueller-Hinton media, and cultured for two days under an aerobic condition at 37° C. Then, the diameters (mm) of antiproliferative circles formed around the respective disks to be tested were measured. The results are shown in Table 3.

TABLE 3
Mueller-Hinton media

| | Extracts prepared to be respective dosages | | | | | | |
|---|---|---|---|---|---|---|---|
| | MH | MH | MH | S | MH + S | MH + S | MH + S |
| Malassezia | 5 mg | 10 mg | 20 mg | 25 mg | 5 mg | 10 mg | 20 mg |
| Antiproliferative | 10 | 12 | 14 | 11 | 16 | 20 | 20 |
| Circles (mm) | | | | | | | |

As is clear from Table 3, the mastic normal-hexane extracts (MH) dose-dependently blocked the proliferation of Malassezia. Moreover, this antifungal activity is enhanced by the addition of squalene of 25 mg/disk.

### Fifth Example

### Antifungal (Malassezia) Activity of Fractionated Ingredients of Mastic Normal-Hexane Extract

The fractions fractionated in the first example were respectively dissolved in acetone. Paper disks for antibiotic assay which are the same as the paper disks for antibiotic assay used in the third example were impregnated with the obtained respective fraction solutions of 5 mg/disk. Moreover, squalene was added to each of the paper disks for antibiotic assay by 25 mg/disk, to prepare disks to be tested.

Then, the respective disks to be tested were vacuum-dried to eliminate the acetone.

Further, the Malassezia prepared to be approximately 10⁶ CFU/ml was applied to the Mueller-Hinton media.

Next, the respective disks to be tested were placed on the Mueller-Hinton media, and cultured for two days under an aerobic condition at 37° C. Then, the diameters (mm) of antiproliferative circles formed around the respective disks to be tested were measured. The results are shown in Table 4.

TABLE 4
Mueller-Hinton media

| | Extracts prepared to be respective dosages | | | | | |
|---|---|---|---|---|---|---|
| | MH | MH | MH | MH | MH | MH |
| Malassezia | Fr. 1 | Fr. 3 | Fr. 5 | Fr. 6 | Fr. 8 | Fr. 10 |
| First time | 25 | 25 | 26 | 21 | 25 | 14 |
| Second | 22 | 25 | 22 | 19 | 24 | 14 |
| time | | | | | | |
| Average | 23.5 | 25 | 24 | 20 | 24.5 | 14 |

As is clear from Table 4, a potent anti-Malassezia activity was confirmed in each combination of squalene and 3-oxo-28-norolean-16,18-diene (Fr. 1), olean-12-en-3,28-dione (or oleanoic aldehyde) (Fr. 3), Tirucallol (Fr. 5-1) and 8-amylin (Fr. 5-2), 3-oxo-17-hydroxy-norolean-12-ene (Fr. 6), and 3-oxo-20-hydroxy-dammar-24-ene (Fr. 8), which are the triterpene ingredients from mastic normal-hexane extracts.

As described above, the antifungal agent of the present invention contains the mastic normal-hexane extracts which are mastic extracts capable of inhibiting the proliferation of fungus (Malassezia) and the squalene capable of inhibiting the proliferation of fungus (Malassezia), and has the enhanced antifungal activity.

Therefore, because the antifungal agent of the present invention has the high antifungal activity and contains the squalene and the mastic normal-hexane extracts which are naturally-derived ingredients, this is safe to use even for the human body.

Then, by the antifungal agent of the present invention, it is possible to perform infection prevention as treatment of Malassezia infections in dogs that cause concern on the medical front.

Further, cleaning of hands and fingers, and affected areas are carried out with ethanol for disinfection, disinfectants, and fungicides on the nursing-care front and medical front. However, a high frequency of cleaning is very likely to damage skin such as causing skin to become rough, stinging wounds, or worsening of dermatitis.

There are disinfectants in which a moisturizing agent such as glycerin, or an emollient agent is contained. However, because its moisturizing effect is weak, and the bactericidal activity is lowered, such disinfectants have been avoided in the nursing-care and medical fronts. Further, such disinfectants do not exert a recovering effect on skin which has already been damaged and cannot become a solution.

On the other hand, because the antifungal agent of the present invention contains the squalene, the antifungal agent is capable of exerting an antifungal effect due to the synergetic effect with the mastic extracts while exerting the moisturizing effect. Therefore, the antifungal agent of the present invention is capable of moisturizing skin for dermatitis, to improve the condition of the skin.

Finally, the respective experimental examples described above are examples of the present invention, and the present invention is not limited thereto. Therefore, it is a matter of course that various modifications according to the design and the like are possible within the range which does not deviate from the technical idea according to the present invention.

1. (canceled)

2. (canceled)

3. (canceled)

4. (canceled)

5. An antifungal agent comprising:
a mastic extract; and
squalene, wherein
the mastic extract is a mastic normal-hexane extract, and
the mastic normal-hexane extract contains at least one of triterpenes of 3-oxo-28-norolean-16,18-diene, 3-oxo-17-hydroxy-norolean-12-ene, and 3-oxo-20-hydroxy-dammar-24-ene, which are respectively expressed by the following chemical formulas 1,5, and 6. [chemical structure]
